(19) United States
(12) Patent Application Publication
(10) Pub. No.: US 2021/0123028 A1
(43) Pub. Date: Apr. 29, 2021

(54) FORMULATION OPTIMIZATION FOR VIRAL PARTICLES

(71) Applicant: Applied Genetic Technologies Corporation, Alachua, FL (US)

(72) Inventors: David R. Knop, Newberry, FL (US); Chantelle Gaskin, Alachua, FL (US); Yuyu Long, Gainesville, FL (US)

(21) Appl. No.: 16/893,724

(22) Filed: Jun. 5, 2020

Related U.S. Application Data
(63) Continuation of application No. PCT/US2018/064062, filed on Dec. 5, 2018.
(60) Provisional application No. 62/594,833, filed on Dec. 5, 2017.

(51) Int. Cl. C12N 7/00; C12N 15/86; A61K 47/18; A61K 47/22; A61K 47/26

(52) U.S. Cl. CPC C12N 7/00; C12N 15/86; A61K 47/183; C12N 2750/14151; A61K 47/26; C12N 2750/14142; C12N 2750/14143; A61K 47/22

(57) ABSTRACT

The present disclosure provides formulations for maintaining viral particles in a stable form. In certain embodiments, the formulation comprises at least one basic amino acid and a buffer solution, wherein the viral particle is present at a concentration of about 1×10¹¹ to about 5×10¹³ DNase resistant virus particles per mL.

### BACKGROUND OF THE INVENTION

Adeno-associated virus (AAV), from the parvovirus family, is a 4.7 kb, replication-defective, non-enveloped animal virus that infects humans and some other primate species. Several features of AAV make this virus an attractive vehicle for delivery of therapeutic proteins by gene therapy, including, for example, that AAV is not known to cause human disease and induces a mild immune response, and that AAV vectors can infect both dividing and quiescent cells without integrating into the host cell genome. AAV offers the capability for highly efficient gene delivery and sustained transgene expression in numerous tissues, including eye, muscle, lung, and brain. AAV has shown promise in human clinical trials.

However, challenges remain in the development of vector formulations to achieve optimal vector safety, stability and efficacy. AAV vectors are potentially susceptible to loss of activity through aggregation, proteolysis and oxidation, as well as through non-specific binding to product contact materials used for vector purification and storage. Aggregation of AAV may result in reduced yield during purification and may have deleterious effects on vector transduction efficiency, biodistribution and immunogenicity following in vivo administration.

The present disclosure provides novel formulations that overcome these challenges and are optimized for viral particles.

### SUMMARY OF THE INVENTION

The present disclosure is based upon the surprising finding of formulations that are optimized for viral particles, in particular AAV particles, that are able to accommodate a high number of DNA resistant particles (DRP)/mL (e.g. about 1×10¹¹ to about 5×10¹³ DRP/mL) and that maintain stability after exposure to a number of harsh conditions. DRP/mL is a measurement of capsid concentration, and capsid concentration is known to drive the aggregation of AAV particles. The present inventions overcome the problem of AAV aggregation.

Accordingly, in a first aspect, the present disclosure features a formulation for maintaining a viral particle in a stable form, the formulation comprising at least one basic amino acid and a buffer solution, wherein the viral particle is present at a concentration of about 1×10¹¹ to about 5×10¹³ DNase resistant virus particles per mL (DRP/mL). In one embodiment, the basic amino acid is selected from the group consisting of arginine, histidine and lysine. In one embodiment, the arginine is present in the formulation in an amount of about 2 mM to about 10 mM. In a further embodiment, the arginine is present in the formulation in an amount of about 5 mM. In one embodiment, the histidine is present in the formulation in an amount of about 2 mM to about 10 mM. In a further embodiment, the histidine is present in the formulation in an amount of about 5 mM. In one embodiment, the lysine is present in the formulation in an amount of about 2 mM to about 10 mM. In a further embodiment, the lysine is present in the formulation in an amount of about 5 mM. In one embodiment, the formulation further comprises a non-ionic surfactant. In a further embodiment, the non-ionic surfactant is tween-20. In another further embodiment, the tween 20 is present in the formulation is an amount of about 0.005% to about 0.025% (v/v). In still another further embodiment, the tween 20 is present in the formulation in an amount of about 0.014% (v/v). In one embodiment, the buffer solution is phosphate buffered saline (PBS). In one embodiment, the viral particle is present at a concentration of about 5×10¹² to about 5×10¹³ DNase resistant virus particles per mL (DRP/mL). In one embodiment, the pH of the formulation is about 7.0 to about 7.5. In a further embodiment, the pH of the formulation is about 7.5. In one embodiment, the viral particle is an adeno-associated virus (AAV). In a further embodiment, the viral particle is a recombinant adeno-associated virus (rAAV). In one embodiment, the formulation is stable after storage at −80 C. In one embodiment, the formulation is stable after heating to 45 C. In one embodiment, the formulation is stable after shaking at high intensity. In one embodiment, the formulation is used for the delivery of a therapeutic. In a further embodiment, the therapeutic is a gene therapy vector.

Other embodiments of the present disclosure are provided infra.

### DETAILED DESCRIPTION OF THE INVENTION

The present disclosure provides novel formulations that are optimized for viral particles, and in particular for AAV viral particles.

### Definitions

In order that the present disclosure may be more readily understood, certain terms are first defined. In addition, it should be noted that whenever a value or range of values of a parameter are recited, it is intended that values and ranges intermediate to the recited values are also intended to be part of this disclosure. Unless otherwise clear from context, all numerical values provided herein can be modified by the term about.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof. Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “about” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “adeno-associated virus” (AAV) as used herein includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). The genomic sequences of various AAV and autonomous parvoviruses, as well as the sequences of the ITRs, Rep proteins, and capsid subunits are known in the art. Such sequences may be found in the literature or in public databases such as the GENBANK database. See, e.g., GenBank Accession Numbers NC 002077, NC 001401, NC 001729, NC 001863, NC 001829, NC 001862, NC 000883, NC 001701, NC 001510, AF063497, U89790, AF043303, AF028705, AF028704, J02275, J01901, J02275, X01457, AF288061, AH009962, AY028226, AY028223, NC 001358, NC 001540, AF513851, AF513852, AY530579, AY631965, AY631966; the disclosures of which are incorporated herein in their entirety. See also, e.g., Srivistava et al., (1983) J. Virol. 45:555; Chiorini et al., (1998) J. Virol. 71:6823; Chiorini et al., (1999) J. Virol. 73:1309; Bantel-Schaal et al., (1999) J. Virol. 73:939; Xiao et al., (1999) J. Virol. 73:3994; Muramatsu et al., (1996) Virology 221:208; Shade et al., (1986) J. Virol. 58:921; Gao et al., (2002) Proc. Nat. Acad. Sci. USA 99:11854; international patent publications WO 00/28061, WO 99/61601, WO 98/11244; U.S. Pat. No. 6,156,303; the disclosures of which are incorporated herein in their entirety.

The term “viral particle” as used herein is meant to refer to viruses, viral particles and viral vectors. Those terms are synonyms and are interchangeable. This term includes wild type viruses, killed, live attenuated, inactivated and recombinant viruses. It further includes virus-based products such as viral vectors, viral particles such as virus-like particles (VLPs) or nucleocapsids. In certain embodiments, the viral particle is from the parvovirus family. In other embodiments, the viral particle is an adeno-associated virus (AAV).

An “AAV viral particle” or “AAV vector particle” or “AAV virus” as used herein refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome such as a transgene to be delivered to a mammalian cell), it is typically referred to as a “rAAV virus” or a “rAAV vector.”

The abbreviation “rAAV” as used herein refers to recombinant adeno-associated virus, also referred to as a recombinant AAV vector (or “rAAV vector”). A “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

As used herein, the term “vector,” “virus vector,” “delivery vector” (and similar terms) as used herein generally refers to a virus particle that functions as a nucleic acid delivery vehicle, and which comprises the viral nucleic acid (i.e., the vector genome) packaged within the virion.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods and materials are now described.

Reference will now be made in detail to preferred embodiments of the disclosure. While the disclosure will be described in conjunction with the preferred embodiments, it will be understood that it is not intended to limit the disclosure to those preferred embodiments. To the contrary, it is intended to cover alternatives, modifications, and equivalents as may be included within the spirit and scope of the disclosure as defined by the appended claims.

### I. Formulations

The present disclosure features optimized formulations for viral particles. In certain aspects, the disclosure features formulations for maintaining a viral particle in a stable form, the formulation comprising a basic amino acid and a buffer solution, wherein the viral particle is present at a concentration of about 1×10¹¹ to about 5×10¹³ DNase resistant virus particles per mL (DRP/mL).

According to one preferred embodiment, a formulation of the present disclosure comprises at least one amino acid selected in the group consisting of arginine, histidine and lysine.

In one embodiment, the basic amino acid is arginine. The formulation can comprise any suitable amount of arginine. In one embodiment, arginine is present in the formulation in an amount of about 2 mM to about 10 mM, for example about 2, about 3, about 4, about 5, about 6, about 7, about 8, about 9, about 10 mM. In one embodiment, arginine is present in the formulation in an amount of about 5 mM.

In one embodiment, the basic amino acid is histidine. The formulation can comprise any suitable amount of histidine. In one embodiment, histidine is present in the formulation in an amount of about 2 mM to about 10 mM, for example about 2, about 3, about 4, about 5, about 6, about 7, about 8, about 9, about 10 mM. In one embodiment, histidine is present in the formulation in an amount of about 5 mM.

In one embodiment, the basic amino acid is lysine. The formulation can comprise any suitable amount of lysine. In one embodiment, lysine is present in the formulation in an amount of about 2 mM to about 10 mM, for example about 2, about 3, about 4, about 5, about 6, about 7, about 8, about 9, about 10 mM. In one embodiment, lysine is present in the formulation in an amount of about 5 mM.

The formulation may further comprise a nonionic surfactant. The nonionic surfactant can be any suitable nonionic surfactant. Nonionic surfactants may include, for example, NP-40, Brij detergents, zwitterionic detergents such as CHAP detergents, octylphenoxypolyethoxy-ethanol (Triton X-100), C12E8, octyl-β-D-glucopyranoside, pluronic surfactants such as Pluronic F68, and polysorbate 20 (tween 20).

The non-ionic surfactant can be present in the formulation in any suitable amount. In one embodiment, the formulation comprises a nonionic surfactant in an amount of about 0.005% to about 0.025% (v/v). In another embodiment, the composition comprises a nonionic surfactant in a concentration of about 0.010-0.020% (v/v). In another embodiment, the composition comprises a nonionic surfactant in a concentration of about 0.010-0.015% (v/v). In another embodiment, the composition comprises a nonionic surfactant in a concentration of about 0.010, 0.011, 0.012, 0.013, 0.014, 0.015, 0.016, 0.017, 0.018, 0.019, 0.020% (v/v). In one embodiment, the composition comprises a nonionic surfactant in a concentration of about 0.014% (v/v).

A preferred nonionic surfactant is polysorbate 20 (tween 20). In one embodiment, the composition comprises tween 20 in an amount of about 0.005% to about 0.025% (v/v). In another embodiment, the composition comprises tween 20 in a concentration of about 0.010-0.020% (v/v). In another embodiment, the composition comprises tween 20 in a concentration of about 0.010-0.015% (v/v). In another embodiment, the composition comprises tween 20 in a concentration of about 0.010, 0.011, 0.012, 0.013, 0.014, 0.015, 0.016, 0.017, 0.018, 0.019, 0.020% (v/v). In one embodiment, the composition comprises tween 20 in a concentration of about 0.014% (v/v).

The formulations of the disclosure comprise a buffer solution. In one embodiment, the buffer solution is phosphate buffered saline (PBS). The formulation can comprise other components. Such other components include, for example, buffers, salts, diluents, pH adjusters, and the like

Formulations of the disclosure are preferably liquid formulations. In other embodiments, the formulations can be a freeze-dried preparation, lyophilized preparation, or other form. With respect to the liquid formulation, in certain embodiments, the liquid formulation is a pharmaceutical formulation. The freeze-dried or lyophilized preparation is typically converted to a liquid form by reconstituting it using a liquid, such as a pharmaceutically acceptable carrier. The pharmaceutically acceptable carrier may be a liquid carrier that contains a buffer and a salt, for instance (i.e., PBS). Examples of suitable buffers and salts, as well as other types of pharmaceutically acceptable carriers, are well known in the art.

pH

A suitable pH for the formulation is any pH at which the viral particle is maintained in a desired state (i.e., viability, infectious titer) over the time period of storage. For example, the pH of the formulation desirably is about 6-9, 6-8.5, 6.5-8.5, 7-8.5, 7.5-8.5, 6-8, 6.5-8, 7-8, 7.5-8, or 7-7.5. In preferred embodiments, the formulation has a pH of about 7.5.

### Stability

The formulations of the present disclosure are stable and can be stored without an unacceptable change in quality, potency, or purity.

The formulation preferably maintains (e.g., preserves) the viral particles such that, over a desired period of time (e.g., about 24 hours, about 48 hours, about 3 days, about 7 days (i.e., about 1 week), about 2 weeks, about 1 month, about 3 months, about 6 months, about 1 year, or about 2 years) at a desired temperature (e.g., about −80 C, about −4 C, about 0° C., about 5° C., about 10° C., about 15° C., about 20° C., about 25° C., about 30° C., about 35° C., about 40° C., about 45° C., or about 50° C.), the stability, infectivity, and/or activity of the viral particles in the composition is not significantly or substantially degraded or is retained to a significant extent (e.g., at least about 50%, at least about 60%, at least about 70%, at least about 80%, at least about 90%, or at least about 95%). In one embodiment, the formulation is stable after storage at −80 C.

In one embodiment, the formulation is stable after heating to 45 C.

In one embodiment, the formulation is stable after shaking at high intensity.

The retention (e.g., maintenance) of viral particle stability is particularly preferred. Viral particle stability, potency (activity) and purity, can be determined by any suitable techniques. Many such techniques are known in the art.

The “stability” of the viral particles refers to the ability of the viral vector particles to maintain structural integrity over time. Suitable techniques for determining viral particle stability include, for example, turbidity analysis by spectrophotometry, determining vector concentration by PCR, determining vector infectivity by cell based assay, and determining purity by protein gel electrophoresis.

The “potency” or “activity” of the viral particles refers to the ability to infect a mammalian cell and express the transgene. Methods to determine potency would include cell based infectivity assays with a PCR read-out (TCDI50) and immunoassays with a spectrophotometric read-out (ELISA).

The “purity” of the viral particles refers to the demonstration of viral particle components existing in the absence of impurities, such as degradation products, co-purified proteins, or other process related products. Methods to demonstrate purity include protein gel electrophoresis, Western blotting, capillary electrophoresis, mass spectrometry, and gel filtration chromatography.

### II. Viral Particles

As described herein, the present disclosure provides novel and inventive formulations for viral particles.

In certain embodiments, the viral particle is present in the formulations at a concentration of about 1×10¹¹ DRP/mL. In certain embodiments, the viral particle is present in the formulation at a concentration of about 5×10¹¹ DRP/mL or more. In certain embodiments, the viral particle is present in the formulation at a concentration of about 1×10¹² DRP/mL or more. In certain embodiments, the viral particle is present in the formulation at a concentration of about 5×10¹² DRP/mL or more. In certain embodiments, the viral particle is present in the formulation at a concentration of about 1×10¹³ DRP/mL or more. In certain embodiments, the viral particle is present in the formulation at a concentration of about 5×10¹³ DRP/mL or more. In certain embodiments, the viral particle is present in the formulation at a concentration of about 1×10¹¹ to about 5×10¹³ DRP/mL.

The viral particle is desirably non-toxic, non-immunogenic, easy to produce, and efficient in protecting and delivering DNA into the target cells. In one particular embodiment, the viral particle is an adeno-associated virus vector (AAV). More than 30 naturally occurring serotypes of AAV are available. Many natural variants in the AAV capsid exist, allowing identification and use of an AAV with properties specifically suited for ocular cells. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

The use of AAVs is a common mode of delivery of DNA as it is relatively non-toxic, provides efficient gene transfer, and can be easily optimized for specific purposes. Among the serotypes AAVs isolated from human or non-human primates (NHP) and well characterized, human serotype 2 is the first AAV that was developed as a gene transfer vector. It has been widely used for efficient gene transfer experiments in different target tissues and animal models. Clinical trials of the experimental application of AAV2 based vectors to some human disease models are in progress, and include such diseases as cystic fibrosis and hemophilia B. Other AAV serotypes include, but are not limited to, AAV1, AAV3, AAV4, AAV5, AAV6, AAV7, AAV8 and AAV9. See, e.g., WO 2005/033321 for a discussion of various AAV serotypes, which is incorporated herein by reference.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from anon-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the disclosure.

In one embodiment, the viral particles (i.e. vectors) useful in formulations described herein contain, at a minimum, sequences encoding a selected AAV serotype capsid protein, or a fragment thereof. In another embodiment, useful viral particles, i.e. vectors, contain, at a minimum, sequences encoding a selected AAV serotype rep protein, or a fragment thereof. Optionally, such vectors may contain both AAV cap and rep proteins. In vectors in which both AAV rep and cap are provided, the AAV rep and AAV cap sequences can both be of one serotype e.g., all AAV2 origin. Alternatively, vectors may be used in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector.

In certain preferred embodiments of the disclosure, the vector is a recombinant adeno-associated virus (rAAV).

The formulations of the disclosure can be administered to the host cell in vitro, in vivo, or ex vivo. In that respect, the host cell can be in a mammal (such as a human), or, for example, part of an organ (such as the heart), or otherwise present in the mammal (such as in a tumor in the mammal). The formulation can contact the host cell by any suitable manner. The formulation can be administered by a variety of routes. Local or systemic delivery can be accomplished by application or instillation into body cavities, by inhalation or insufflation of an aerosol, or by parenteral introduction, comprising intermuscular, intramuscular, intravenous, intraperitoneal, intraocular, transtympanical, transdermal, internasal, or subcutaneous administration, or by other means. The composition can be delivered to a specific tissue, organ, gland, or other part of a human patient's body (e.g., a tumor, a limb such as the leg, the lungs, the brain, the eye, or the ear).

In certain embodiments, the disclosure provides that the formulations are used for the delivery of a therapeutic. In one embodiment, the therapeutic is a gene therapy vector.

A number of embodiments of the disclosure have been described. Nevertheless, one skilled in the art, without departing from the spirit and scope of the disclosure, can make various changes and modifications of the disclosure to adapt it to various usages and conditions. Accordingly, the following examples are intended to illustrate, but not limit, the scope of the invention claimed.

### Examples

### Example 1. Degradation Study

The goal of this study is to compare different formulations for AAV particles and to optimize a formulation that can accommodate a high number of DNA resistant particles (DPR)/mL and maintain stability after exposure to harsh conditions.

### Study Design

The following experimental formulations shown in Table 1 were tested under the following conditions: (1) Freeze/thaw, (2) heat (45 C) and (3) agitation.

TABLE 1

| | Description | Titer | Storage |
|---|---|---|---|
| | of buffer | DRP/ml | Temperature |
| Formulation 1 | 5 mM Arginine, | 9.22E+12 | 4 |
| | 1X PBS, | | |
| | 0.014% Tween-20, | | |
| | pH 7.5 | | |
| Formulation 2 | 5 mM Histidine, | 2.97E+13 | 4 |
| | 1X PBS, | | |
| | 0.014% Tween-20, | | |
| | pH 7.5 | | |
| Formulation 3 | 5 mM Arginine, | 1.45E+13 | −80 |
| | 1X PBS, | | |
| | 0.014% Tween-20, | | |
| | pH 7.5 | | |
| Control | | 5.22E+12 | −80 |

### Turbidity Test

Freeze/Thaw Run

TABLE 2

| | | | | | Total sample |
|---|---|---|---|---|---|
| | Sample Load | Buffer Load | Work Conc. | Work volume/ | load volume, |
| Sample | volume/well ml | volume/well ml | DRP/ml | well ml | ul (Triplicate) |
| Form 1 | 0.20 | 0.00 | 9.22E+12 | 0.2 | 600 |
| Form 2 | 0.06 | 0.14 | 9.22E+12 | 0.2 | 180 |
| Form 3 | 0.13 | 0.07 | 9.22E+12 | 0.2 | 390 |
| Control | 0.20 | 0.00 | 5.22E+12 | 0.2 | 600 |
| Blank 1 | 0.00 | 0.20 | 0 | 0.2 | 0 |
| (1X BSST) | | | | | |
| Blank 2 | 0.00 | 0.20 | 0 | 0.2 | 0 |
| (1X PBS) | | | | | |

The samples listed in Table 2 are loaded into a 48-well flat-bottom plate in triplicates. Optical density reading is measured at a wavelength of 600 nm (OD600) at room temperature, without shaking. All work concentrations were adjusted to be the same concentration. This allows the comparison of datasets across the three formulations (Form 1, Form 2, Form 3) and control. The plate is then frozen at −80 C and left overnight in a freezer. The plate is then taken out of the freezer the following morning, and thawed at room temperature before testing. Another reading at OD600 will be obtained after the samples are completely warmed up to room temperature.

Heating Run

TABLE 3

| | | | | | Total sample |
|---|---|---|---|---|---|
| | Sample Load | Buffer Load | Work Conc. | Work volume/ | load volume, |
| Sample | volume/well ml | volume/well ml | DRP/ml | well ml | ul (Triplicate) |
| Form 1 | 0.20 | 0.00 | 9.22E+12 | 0.2 | 600 |
| Form 2 | 0.06 | 0.14 | 9.22E+12 | 0.2 | 180 |
| Form 3 | 0.13 | 0.07 | 9.22E+12 | 0.2 | 390 |
| Control | 0.20 | 0.00 | 5.22E+12 | 0.2 | 600 |
| Blank 1 | 0.00 | 0.20 | 0 | 0.2 | 0 |
| (1X BSST) | | | | | |
| Blank 2 | 0.00 | 0.20 | 0 | 0.2 | 0 |
| (1X PBS) | | | | | |

The samples listed in Table 3 are loaded into a 48-well flat-bottom plate in triplicates. All work concentrations were adjusted to be the same concentration. This allows the comparison of datasets across the three formulations (Form 1, Form 2, Form 3) and control. The plate is inserted into plate reader SpectraMax i3x, which is pre-heated to 45 C. The plate is kept in the plate reader for 30 min at 45 C, without shaking. Next, a reading at OD600 is obtained at 45 C, without shaking.

Agitation Run

TABLE 4

| | | | | | Total sample |
|---|---|---|---|---|---|
| | Sample Load | Buffer Load | Work Conc. | Work volume/ | load volume, |
| Sample | volume/well ml | volume/well ml | DRP/ml | well ml | ul (Triplicate) |
| Form 1 | 0.20 | 0.00 | 9.22E+12 | 0.2 | 600 |
| Form 2 | 0.06 | 0.14 | 9.22E+12 | 0.2 | 180 |
| Form 3 | 0.13 | 0.07 | 9.22E+12 | 0.2 | 390 |
| Control | 0.20 | 0.00 | 5.22E+12 | 0.2 | 600 |
| Blank 1 | 0.00 | 0.20 | 0 | 0.2 | 0 |
| (1X BSST) | | | | | |
| Blank 2 | 0.00 | 0.20 | 0 | 0.2 | 0 |
| (1X PBS) | | | | | |

The samples listed in Table 4 are loaded into a 48-well flat-bottom plate in triplicates. All work concentrations were adjusted to be the same concentration. This allows the comparison of datasets across the three formulations (Form 1, Form 2, Form 3) and control. The plate is inserted into plate reader SpectraMax i3x. Shaking is set at high intensity for 5 min in an orbital/linear way before measuring its absorbance at OD600. Reading at OD600 at R.T. is obtained immediately after shaking.

### SDS-PAGE Silver Staining

Experimental Plan

The three conditions (freeze/thaw, heat (45° C.) and agitation) are tested with four samples for each condition (formulation 1 (Form 1), formulation 2 (Form 2), formulation 3 (form 3) and Control). All formulations and control are tested with 3.0×10⁹ DRP/well.

Sample Preparation

According to the silver staining procedure, samples need to be submitted at a concentration of no less than 2.5×10¹¹ DRP/ml, 12 ul/well.

Experimental Procedure

Tested samples from the turbidity test will be immediately subject to silver staining. Briefly, viral samples are mixed with a buffer containing dyes and denaturing agents. The dentatured test articles are loaded into protein electrophoresis gel wells alongside a positive control and molecular weight ladder. The gel is subjected to electrophoresis for separation of proteins by size. The gel is then fixed, stained and developed for imaging SOP

SV40-qPCR

Briefly, viral samples and a positive control are subjected to DNAse and proteinase treatment followed by an enzyme inactivation step. Test articles are mixed with a buffer containing primers and a fluorescent probe specific to the SV40 sequence in the viral genome. PCR is used to amplify and detect the viral DNA, and the test articles are quantified against a DNA standard curve.

### TCID₅₀

The results of the three formulations in the three conditions from the turbidity test and SDS-PAGE test will be evaluated and compared with control (1×BSST). One or two formulations that show the best stability will be scheduled for a TCID₅₀ test to evaluate its infectivity, after treated with the selected conditions (freeze/thaw, heating or shaking).

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

Each reference, patent and patent application referred to in the instant application is hereby incorporated by reference as if each reference were noted to be incorporated individually.

1. A formulation for maintaining a viral particle in a stable form, the formulation comprising at least one basic amino acid and a buffer solution, wherein the viral particle is present at a concentration of about 1×10¹¹ to about 5×10¹³ DNase resistant virus particles per mL (DRP/mL).

2. The formulation of claim 1, wherein the basic amino acid is selected from the group consisting of: arginine, histidine and lysine.

3. The formulation of claim 2, wherein the arginine is present in the formulation in an amount of about 2 mM to about 10 mM.

4. The formulation of claim 3, wherein the arginine is present in the formulation in an amount of about 5 mM.

5. The formulation of claim 2, wherein the histidine is present in the formulation in an amount of about 2 mM to about 10 mM.

6. The formulation of claim 5, wherein the histidine is present in the formulation in an amount of about 5 mM.

7. The formulation of claim 2, wherein the lysine is present in the formulation in an amount of about 2 mM to about 10 mM.

8. The formulation of claim 7, wherein the lysine is present in the formulation in an amount of about 5 mM.

9. The formulation of claim 1, further comprising a non-ionic surfactant.

10. The formulation of claim 1, wherein the non-ionic surfactant is tween-20.

11. The formulation of claim 10, wherein the tween 20 is present in the formulation is an amount of about 0.005% to about 0.025% (v/v).

12. The formulation of claim 11, wherein the tween 20 is present in the formulation in an amount of about 0.014% (v/v).

13. (canceled)

14. The formulation of claim 1, wherein the viral particle is present at a concentration of about 5×10¹² to about 5×10¹³ DNase resistant virus particles per mL (DRP/mL).

15. The formulation of claim 1, wherein the pH of the formulation is about 7.0 to about 7.5.

16. The formulation of claim 15, wherein the pH of the formulation is about 7.5.

17. The formulation of claim 1, wherein the viral particle is an adeno-associated virus (AAV).

18. The formulation of claim 17, wherein the viral particle is a recombinant adeno-associated virus (rAAV).

19. The formulation of claim 1, wherein the formulation is stable after storage at −80 C or the formulation is stable after heating to 45 C.

20. (canceled)

21. (canceled)

22. The formulation of claim 1, wherein the formulation is used for the delivery of a therapeutic.

23. The formulation of claim 22, wherein the therapeutic is a gene therapy vector.
